(19) United States
(12) Patent Application Publication
(10) Pub. No.: US 2015/0118224 A1
(43) Pub. Date: Apr. 30, 2015

(54) ENDOTHELIAL CELLS ACTIVATION BIOMARKERS CHARACTERIZING ANTIBODY MEDIATED REJECTION AND USES THEREOF

(71) Applicants: ASSISTANCE PUBLIQUE HÔPITAUX DE PARIS, Paris (FR); UNIVERSITE PIERRE ET MARIE CURIE, Paris Cédex 05 (FR)

(72) Inventors: Yi-Chun Xu-Dubois, Paris (FR); Eric Rondeau, Vincennes (FR); Julie Peltier, Paris (FR); Alexandre Hertig, Paris (FR); Isabelle Brocheriou, Paris (FR)

(21) Appl. No.: 14/390,751

(22) PCT Filed: Apr. 4, 2013
(86) PCT No.: PCT/IB2013/000867

§ 371 Date: Oct. 3, 2014

Related U.S. Application Data
(60) Provisional application No. 61/619,986, filed on Apr. 4, 2012.

(51) Int. Cl. G01N 33/68; A61K 39/395; C07K 16/18; C07K 16/28; A61K 38/05

(52) U.S. Cl. CPC G01N 33/6893; C07K 16/2887; A61K 38/05; C07K 16/18; A61K 39/3955; G01N 2333/47; G01N 2333/8121; G01N 2800/245
USPC 424/133.1; 424/143.1; 514/21.91; 424/172.1; 436/501; 435/6.11; 435/6.12; 506/7; 506/9; 506/18; 435/7.92

(57) ABSTRACT

Described herein are methods and kits for the detection of endothelial cell injury and/or activation and to the diagnostic of transplant antibody mediated rejection (ABMR). The invention further relates to methods and kits for diagnosing endothelial to mesenchymal transition (EndMT). In various embodiments, the methods comprise assessing expression of one, two or three biomarkers selected from Fascin1, Vimentin and Hsp47.

### FIELD OF THE INVENTION

The invention relates to the field of medicine, and more particularly to the detection of endothelial cell injury and/or activation and to the diagnostic of transplant antibody mediated rejection.

### BACKGROUND OF THE INVENTION

In transplantation, the antibody mediated rejection (ABMR) is currently a major thread for the long term graft survival [1-3]. Although important advances have been made in the detection of preexistent donor specific antibodies (DSA) and the use of desensitization protocols before transplantation avoids efficiently the hyperacute form of ABMR, the early/acute ABMR developed in the cross-match positive, but desensitized patients still occurs; and the late/chronic ABMR because of the presence of de novo DSA or the increased activity of low preexisting title of DSA now emerges as a leading cause of late graft loss. This form of ABMR is usually at beginning indolent, but it destroys progressively the graft structure. When the graft function starts to be altered, the pharmaceutical intervention often has no efficiency because the chronic ABMR associated graft fibrosis is too extensive and is no longer reversible. However the diagnosis of the ABMR remains still difficult and mainly based on a triad of criteria: 1) the presence in the recipient's plasma of donor specific antibodies (DSA) for HLA antigens; 2) inflammation in the micro-circulation of the allograft such as peri-tubular capillaritis (ptc) and glomerulitis; and 3) deposits of C4d (a fragment of complement) on the endothelial cells of peritubular capillaries.

Vascular endothelial cells of an allograft are the major target of the anti-donor specific antibodies (DSA), in vitro and in vivo experiences [4-7] as well as human renal transplantation research data [8] showed that the ligation of HLA molecules on the vascular endothelial cells with corresponding antibodies and/or activation of complement can trigger cell stress response and by various signaling pathways it can affect the endothelial transcriptional profile and induce an up-regulation of molecules involved in inflammation, coagulation, cell motility and endothelial repair, a process reminiscing EndMT.

Endothelial to mesenchymal transition (EndMT) is a process comparable to Epithelial to mesenchymal transition (EMT) which is a crucial process during embryo development, cancer progression, tissue repairing and/or fibrogenesis. During this process, the epithelial cells undergo a molecular switch from a polarized epithelial phenotype to a highly motile, non-polarized mesenchymal phenotype including the expression of large amounts of molecules involved in cell motility which is the necessary mechanism for dispersing cells in embryo during development, initiating metastasis of epithelial cancer cells and forming interstitial fibroblasts cells in injured adult tissues. In renal allografts, it has been reported that the epithelial phenotypic changes, the first steps of EMT, in the tubular epithelial cell was associated with ongoing renal allograft injury [9-11].

EndMT has been also described as an important fibrogenic process by which the endothelial cells of capillaries could be the source of the myofibroblasts during the progression of the diseases in heart and in kidney [12-14]. However, the detection of EndMT, especially of the expression of mesenchymal markers such as fascin1, vimentin, or hsp47 on micro-vascular endothelial cells has never been suggested as an indicator of endothelial cell injury or activation for detecting acute and/or chronic ABMR of an allograft or other micro vascular endothelial injury related diseases.

Fascin1 is an actin-bundling protein. Cross-linking of actin filament by fascin1 is essential for the formation of lamellipodia or filopodia, which are important structures for cell motility during embryogenesis and cancer metastasis. The high level of fascin1 expression is detected at the invasive front of cancers of different cellular origin. Its expression is in contrast down-regulated when tumor cells reach their metastatic destination and stop migrating. Thus fascin1 promotes and reflects a motile phenotype in epithelial cells. In addition, an intense expression of fascin1 was correlated with the increased histological grade and a poor survival of patients with aggressive carcinomas from many tissues [15]. So far, no study has reported fascin1 expression in kidney diseases, let alone its possible use as a biomarker of ABMR, EndMT or cell injury and/or activation.

Vimentin is an intermediate filament protein expressed only in mesenchymal cells and is now regarded as a canonical marker of epithelial to mesenchymal transition. In addition to its interest as a mesenchymal cell marker, recent papers have shown that vimentin can contribute to EMT via the up-regulation of the expression of several EMT-linked genes [16]. However, vimentin has never been suggested for detecting EndMT, as an indicator of endothelial cell injury or activation or as biomarker for diagnosing ABMR.

Hsp47 is a well-known stress protein acting as a collagen-specific chaperone in the folding and the assembly of pro-collagen molecules and is not expressed in endothelial or epithelial cells of normal tissue [17]. However, hsp47 has never been suggested for detecting EndMT, as an indicator of endothelial cell injury or activation or as biomarker for diagnosing ABMR.

De novo expression of hsp47 and vimentin was observed in tubular epithelial cells in animal models with kidney injury [18], or in diseased human kidney and in renal transplants [9,19]. However, the expression of hsp47 and vimentin in vascular endothelial cells was not studied in human renal diseases. Mahesh et al. have shown the expression of vimentin in leukocytes and in some endothelial cells of rat cardiac allografts after the injection of a specific antibody against vimentin [20]. Ohba et al. have shown hsp47 expression in interstitial cells but not in endothelial cells in the renal allografts with ABMR [21].

The medical community stresses the necessity of developing new biomarkers in diagnosis, grading and staging of ABMR [22] because the current criteria are neither necessary nor sufficient: The presence of DSA in the plasma of patients is a pre-requisite for the diagnosis of ABMR, but it is not always associated with ABMR and we still don't know the biological significance of low alloantibody levels detected by the very sensitive method Luminex™. In addition, some antibodies may be present in the context of accommodation that is without significant harmful effect even if C4d is detected. In contrast, some anti-donor antibodies directed against MICA or MICB antigens or other anti-endothelial cell antibodies [23-25] are not included in the panel of DSA detection. The presence of C4d on the peritubular capillaries is a good witness of complement activation thus strongly suggestive of ABMR. Yet, recent studies pointed out its poor sensitivity as a diagnostic tool, since it can detect only 50-60% of ABMR patients [8,26]. Currently, the presence of inflammatory cells in microcirculation such as capillaritis and glomerulitis is considered as a strong indicator of ABMR which obviously reflects antibody mediated graft injury. However, a small amount of inflammatory cells in the capillaries may be difficult to be assessed by routine morphological analysis. Even though, the clinical significance of a small amount of inflammatory cells in the microcirculation is not yet established and it is likely that many patients with mild ABMR are underdiagnosed and not appropriately treated.

Early and accurate diagnosis of these two forms of ABMR is thus important because a specific treatment, different from that of T cell mediated rejection, can be proposed for these patients including plasma exchanges, Mg, anti-CD20 antibodies (rituximab) and proteasome inhibitor (bortezomib). Otherwise, in the absence of appropriate treatment, the patients can lose their graft rapidly. In addition, when the diagnostic can be made with certainty, undue over-immunosuppression and high level expenses will be prevented for the patients who needn't.

There is further a need for methods allowing the diagnosis of endothelial injuries in the diseased organs other than transplants and for direct endothelial cell activation markers which can be used in the clinic.

The present invention addresses these needs, as it relates to methods, biomarkers, kits and treatment approaches useful in the diagnosis, grading and staging of endothelial cell injury, which is useful in the diagnosis and prevention of development and progression of antibody mediated transplant rejection, as well as other solid organ diseases associated with endothelial cell injury.

Additional features of the invention will be apparent from review of the disclosure, figures, and description of the invention below.

### BRIEF SUMMARY OF THE INVENTION

According to a first aspect, the invention is concerned with a method for detecting vascular endothelial cell injury and/or activation in a mammalian subject. The method comprises detecting endothelial to mesenchymal transition (EndMT) and EndMT is indicative of presence of cell injury and/or activation. In various embodiments, detecting EndMT comprises assessing expression of one, two or three biomarkers selected from the group consisting of Fascin1, Vimentin and Hsp47.

According to a second aspect, the invention is concerned with a method for diagnosing antibody mediated rejection (ABMR) in an allograft. The method comprises assessing expression level of at least one biomarker selected from the group consisting of Fascin1 Vimentin and Hsp47 and wherein the expression level is indicative of the presence or absence of ABMR in the allograft. In some embodiments the allograft is a sensitized allograft. In some embodiment, the method further comprises the step of excluding recurrent diseases and/or other diseases possibilities such as excluding alto-antibody independent vascular diseases such as thrombotic microangiopathy or small vessel vasculitis.

According to a third aspect, the invention is concerned with a method for identifying a mammalian subject showing endothelial injury. The method comprises assessing in endothelial cells expression level of at least one biomarker selected from the group consisting of Fascin1, Vimentin and Hsp47, and wherein said expression level is indicative of the presence or absence of endothelial injury.

According to a fourth aspect, the invention is concerned with a method for preventing progression of antibody mediated tissue injury in a subject with an allograft. The method comprises measuring in the allograft of the patient expression of at least one biomarker selected from the group consisting of Fascin1, Vimentin and Hsp47, wherein the expression is indicative of an endothelial injury binding of donor specific antibodies (DSA) to endothelial cells (and/or consecutive to related cellular consequences); and reducing the level and/or the production of said DSA. Preferably, the method further comprises protecting the allograft against antibody mediated tissue injury. In some embodiments, the expression is indicative of an endothelial injury consecutive to binding of donor specific antibodies (DSA) which engage a cell response. In other embodiments, the expression is indicative of endothelial cell reaction to the binding of DSA. The method may further comprises protecting the allograft against further injury such as complement activation and/or other antibody mediated graft injury. In some embodiments, the method comprises protecting the allograft through complement inactivation, for instance by administration of anti-C5 antibodies to the engrafted subject.

According to a further aspect, the invention is concerned with a non-invasive method for the detection of EndMT markers, including but not limited to detection in the blood and/or urine of an engrafted recipient.

The invention further relates to a kit for diagnosing endothelial to mesenchymal transition (EndMT) during acute and/or chronic antibody mediated rejection (ABMR) of an allograft. The kit comprises a combination of antibodies specific for at least two biomarkers selected from the group consisting of Fascin1, Vimentin and Hsp47. In a preferred embodiment, the kit is optimized for immunohistochemistry and it further comprises components for immunohistochemistry visualization.

An advantage of the invention is that it provides means and biomarkers suitable and precisely for direct detection of endothelial cell injury due to ABMR and the other micro-vascular endothelial injury related diseases.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order that the invention may be readily understood, embodiments of the invention are illustrated by way of example in the accompanying figures.

FIG. 1 is a panel of pictures showing EndMT marker expression (fascin1, vimentin and hsp47) in the normal kidney (A1, B1, C1) and in the renal graft with acute ABMR (A2, B2, C2). FIG. 1-A1: No vimentin expression in the peritubular capillary endothelial cells in Normal kidney; FIG. 1-A2: Intense vimentin expression in the peritubular capillary endothelial cells in Renal allograft with acute ABMR; FIG. 1-B1: Limited peri-nuclear expression of fascin in the glomerular and peritubular capillary endothelial cells in Normal kidney; FIG. 1-B2: Intense cytoplasmic fascin expression in the peritubular capillary endothelial cells in Renal allograft with acute ABMR; FIG. 1-C1: No hsp47 expression in the peritubular capillary endothelial cells in Normal kidney; FIG. 1-C2: Intense hsp47 expression in the peritubular capillary endothelial cells in Renal allograft with acute ABMR.

FIG. 2 is a bar graph showing levels of EndMT marker expression (fascin1, vimentin and hsp47) in endothelial cells of peritubular capillaries in normal allografts, in the allografts with acute cell mediated rejection (CMR) or in the allografts with acute ABMR (aABMR) and chronic ABMR (cABMR).

FIG. 3 is a bar graph comparing of endothelial expression level of EndMT markers (fascin1, vimentin and hsp47) in the allograft in presence or absence of peritubular deposition of C4d.

FIG. 4 is a bar graph comparing endothelial expression level of EndMT markers (fascin1, vimentin and hsp47) in the patients according to the presence or absence of anti-donor specific HLA (class I or II) antibodies (DSA) in their plasma.

FIG. 5A is a panel of pictures showing expression of EndMT markers (fascin1, vimentin and hsp47) in the endothelial cells of peritubular and glomerular capillaries of renal graft of a patient who was not diagnosed as ABMR at time of biopsy.

FIG. 5B is a panel of pictures showing evident ABMR lesions in a subsequent biopsy of the renal transplanted patient referred to in FIG. 5A.

FIGS. 6A and 6B are line graphs showing the different time courses of graft function (FIG. 6A) or proteinuria (FIG. 6B) according the presence or absence of EndMT marker expression at time of biopsy or 1, 2, 3 or 4 years post biopsy in the patients who were not diagnosed as ABMR at time of biopsy.

FIGS. 7A and 7B are line graphs illustrating ROC curve analyses and showing the value of EndMT marker (FIG. 7A) or of peritubular capillaries (ptc) (FIG. 7B) for the diagnosis of ABMR.

FIGS. 8A and 8B are line graphs illustrating different time course of graft function (FIG. 8A) or proteinuria (FIG. 8B) according the presence or absence of EndMT marker expression in the peritubular capillary cells, at time of biopsy or 1, 2, 3 or 4 years post biopsy in the patients with ptc.

FIG. 9 is a line graph illustrating ROC curve analysis in the second setting of 74 biopsies confirming an excellent value of EndMT marker for the diagnosis of ABMR.

### DETAILED DESCRIPTION OF PREFERRED EMBODIMENTS

In the following description of the embodiments, references to the accompanying drawings are by way of illustration of an example by which the invention may be practiced. It will be understood that other embodiments may be made without departing from the scope of the invention disclosed.

### a) General Overview of the Invention

To date, the diagnosis of the ABMR is difficult and mainly based on a triad of criteria: (1) the presence in the recipient's blood of antibodies specific for the donor HLA antigens (DSA); (2) inflammation in the micro-circulation of the allograft (named peri-tubular capillaritis and glomerulitis); and (3) deposits of C4d (a fragment of complement) on the endothelial cells of peritubular capillaries.

With regard to DSA, their presence is a pre-requisite for the diagnosis of ABMR. However it does not necessarily indicate ABMR. Indeed, some antibodies may be present in the context of accommodation. In contrast, absence of detectable of DSA in some ABMR patients can be due to absorption of DSA by the rejected allograft, thus DSA becomes undetectable in the plasma, or, due to the anti-endothelial cell antibodies which are not included in the present panel of DSA detection.

Currently peri-tubular capillaritis and glomerulitis are recognized as strong indicators of ABMR in clinic, because endothelial cells are the main target of DSA, and the presence of inflammatory cells within the capillaries obviously reflects ongoing injury. There are however some limitations to use capillaritis as a diagnostic tool for ABMR: although the presence of inflammatory cells within the capillaries can be the consequence of endothelial cell activation during ABMR, a recent paper has report that the capillaritis in early biopsies was often allo-antibody independent [27]. And as an indirect evidence of endothelial cell activation, it may present in a relative late stage of disease; last, a small amount of inflammatory cells in the capillaries may be difficult to be assessed by routine morphology analysis or to be evaluated as the evidence for the diagnosis of ABMR. Consequently, the proportion of patients diagnosed for ABMR using the capillaritis as criterion could be like the tip of iceberg.

Last, the presence of C4d on the peri-tubular capillaries is clearly suggestive of ABMR, yet it is poorly sensitive as a diagnostic tool because in the most recent series at least 40% of patients with ABMR were C4d negative.

The inventors have discovered direct biomarkers of endothelial cell injury for the diagnosis of ABMR which are much more sensitive than the existing indirect markers. These direct biomarkers are Fascin1, Vimentin and Hsp47, and they can be used separately or in combination of two or three.

More particularly, the inventors have found that endothelial to mesenchymal phenotypic switch or endothelial to mesenchymal transition (EndMT) can be used to reflect the degree of endothelial activation in patients who developed donor specific antibodies (DSA). Such information can then be used to increase the capacity of the pathologists or clinicians to diagnose acute and chronic ABMR of an allograft in mammalian subjects.

After studying a plurality of biomarkers, the inventors have found that, at least in the case of a renal allograft, the combination of Fascin1, Vimentin and HSP47 expressed by endothelial cells of peri-tubular capillaries provides the best combination for diagnosis purposes. This discovery thus supports many diagnostic and detection methods for use in the clinic, kits therefor, as well as new therapeutic methods for preventing the progression of antibody mediated tissue injury of various types of allografts.

### B) Diagnostic Methods

As indicated hereinbefore and exemplified hereinafter, one aspect of the invention concerns methods and at least one biomarker selected from Fascin1, Vimentin and Hsp47: (i) for detecting endothelial cell injury and/or activation in a mammalian subject; (ii) for diagnosing acute and/or chronic antibody mediated rejection (ABMR) in an allograft; (iii) for identifying a mammalian subject showing vascular endothelial injury related diseases; and/or (iv) for preventing the progression of antibody mediated tissue injury in a sensitized subject with an allograft. The invention also encompasses all possible particular combinations of two or three biomarkers such as: Fascin1 and Vimentin; Fascin1 and Hsp47; Vimentin and Hsp47; and the combination of Fascin1, Vimentin and Hsp47.

According to a first aspect, the invention relates to a method for detecting endothelial cell injury and/or activation in a mammalian subject. In one particular embodiment, the method comprises detecting endothelial to mesenchymal transition (EndMT) and, according to this method, EndMT is indicative of presence of endothelial cell injury and/or activation.

In one embodiment, the endothelial cell injury and/or activation is detected in a sensitized allograft (i.e. with the presence of DSA) and is indicative of an antibody mediated rejection (ABMR) of the allograft. In another embodiment the tissue is a solid organ and the endothelial cell injury and/or activation is indicative of any one of small-vessel vasculitis, thrombotic microangiopathy, and/or anti-phospholipid syndrome.

According to a further aspect, the invention relates to a method for diagnosing antibody mediated rejection (ABMR) in a sensitized allograft. In one particular embodiment, the method comprises assessing expression levels of a combination of biomarkers consisting of Fascin1, Vimentin and Hsp47 and, according to this method, these expression levels are indicative of the presence or absence of ABMR in said allograft.

According to another aspect, the invention relates to a method for identifying a mammalian subject showing endothelial injury. In one particular embodiment, the method comprises assessing in endothelial cells expression levels of a combination of biomarkers consisting of Fascin1, Vimentin and Hsp47.

As used herein the term “subject” includes complex living organisms susceptible to cell injury, and more particularly living organisms in which acute or chronic humoral rejection (i.e. acute and/or chronic ABMR) can occur. The term “subject” includes animals such as mammals. Preferably, the subject is a mammal, including, but not limited to, species such as a human, a dog, a cat, a horse, a bovine, a rabbit, a rat, a mouse, and wild animals living in zoos (e.g. lion, tiger, elephant, panda, bear, etc.). More preferably, the subject is a human. Even more preferably, the subject is a human patient in need of treatment, including but not limited to, an engrafted human patient who has received an allograft.

As used herein the term “allograft” refers to a surgical transplant of tissue between genetically different individuals of the same species. The term allograft excludes isografts and xenografts. The term allograft encompasses any solid organ transplant such as a renal allograft, a heart allograft, a lung allograft, a liver allograft, a pancreas allograft, an intestine allograft and/or a body member allograft (e.g. hand, feet, leg, etc). It also includes other allografts such as muscle allograft, face engraft, eyes, etc.

Accordingly, the invention also relates to a diagnostic method for detecting endothelial cell injury and/or activation in a human patient having an allograft. In one embodiment the method comprises:
obtaining a biological sample from said human patient; and
detecting endothelial to mesenchymal transition (EndMT);
   wherein presence of EndMT is indicative of cell injury and/or activation.

In one particular embodiment, detecting EndMT comprises assessing expression in the allograft of at least one biomarker (preferably two and more preferably three biomarkers) selected from Fascin1, Vimentin and Hsp47. In one embodiment, the biological sample is a biopsy of the allograft. In another embodiment, the biological sample is a urine sample or a plasma sample.

The invention further relates to a diagnostic method for detecting an antibody mediated rejection (ABMR) in a human patient having an allograft. In one embodiment the method comprises:
obtaining a biological sample from said human patient; and
assessing expression of at least one biomarker (preferably two and more preferably three biomarkers) selected from the group consisting of Fascin1, Vimentin and Hsp47;
   wherein said expression level is indicative of the presence or absence of ABMR in said allograft.

In one embodiment, the biological sample is a biopsy of a renal allograft, a heart allograft, a lung allograft, a liver allograft, a pancreas allograft and/or an intestine allograft. In another embodiment, the biological sample is a urine sample or a plasma sample.

In other embodiments, the subject may be afflicted by a disease or condition directly or indirectly related to the production of donor specific antibodies (DSA), a virus infection, a toxin aggression, coagulation problem, or auto-inflammatory diseases.

In some embodiments, detecting EndMT comprises assessing expression of one, two or three biomarkers consisting of Fascin1, Vimentin and Hsp47. In specific embodiments, the endothelial to mesenchymal transition (EndMT) is shown by an upregulation of fascin1 and de novo expression of vimentin and hsp47 in the endothelial cell of peritubular and/or glomerular capillaries.

Increased expression levels for each of Fascin1, Vimentin and Hsp47 may be detected compared to control values. In some embodiments, the control values are expression levels (i.e. average value) in normal kidney samples obtained from the healthy part of kidneys from nephrectomies performed due to renal carcinoma or in subjects without endothelial cells-related disease. For instance, in the examples, the level of fascin1, vimentin and hsp47 expression in endothelial cells was semi-quantified from the proportion of peritubular capillaries displaying positive staining: 0: none, 1: <10%; 2: 10% to 24% 3: 25% to 50%, 4: >50%. Equal or more than 10% of peritubular capillaries showing fascin1, vimentin or hsp47 was defined as EndMT positive allograft.

Expression of the biomarkers of the invention may be assed using any suitable method or technique. In the examples, the expression of fascin1, vimentin and hsp47 was assessed by immunohistochemistry on paraffin tissue in using specific antibodies against fascin1, vimentin or hsp47. The immunoreactive proteins were visualized using Envision™+HRP system (AEC) (DakoCytomation™). Immunohistochemistry is preferred to ascertain that the expression of these markers is in endothelial cells. In situ hybridization or rtPCR for detecting mRNA is also conceivable, yet likely much less economical in terms of money and time required.

It is also conceivable to detect only peptidic fragments of fascin1, vimentin and hsp47. It is also conceivable to detect expression of these biomarkers at the molecular or nucleic acid level (e.g. mRNA or protein) by suitable methods and techniques such as RT-PCT, in situ hybridization, immunoblotting, or flow cytometric analysis etc.

In addition, the invention encompasses non-invasive methods of detection and one may envision assessing expression of one, two or the three biomarker(s) fascin1, vimentin and hsp47 in biological fluids (e.g. urine, plasma, etc.) as summarized in Example 3. The level measured in a biological fluid could be compared to the level of the biomarker(s) in patients with ABMR and/or be compared with the level of the biomarker(s) in patients with a normal allograft.

As indicated herein before, the principles of the invention are applicable to humans and to other mammalian species. Table 1 hereinafter provides relevant information about biomarkers of the invention in different mammalian species.

TABLE 1
GenBank ™ Accession numbers (Gene ID) for Fascin1, Vimentin
and Hsp47

| Species | Fascin1 | Vimentin | Hsp47 |
|---|---|---|---|
| Human | 6624 | 7431 | 871 |
| dog | 489880 | 477991 | |
| bovine | 507342 | 280955 | |
| equine | | 100056088 | |

In the setting of transplantation, it is envisioned that that ABMR of non-renal allografts would as well be diagnosed using the present invention because the microvascular damage induced by anti-donor HLA antibodies is broad and not organ-specific. In addition, there are pathologies that will injure the native endothelium (e.g. a vasculitis, a thrombotic microangiopathy), suggesting applicability of the invention to various transplanted or non transplanted organs or tissues. Accordingly, invention encompasses various types of allografts including, but not limited to, a renal allograft, a heart transplant, a lung allograft, a liver allograft, a pancreas allograft, an intestine allograft, a body member allograft, a muscle allograft, a face allograft and other body parts such. As used herein, “engrafted” recipient or “engrafted” human patient, refers to a subject whom has been the subject of a an allograft or transplantation.

According to the preferred embodiments, expression of the three biomarkers according to the invention are assessed or used in combination. As used herein, the term “combination” refers to a test or method where the three biomarkers are assessed together. As used herein, “combination” also encompasses tests or methods where the three biomarkers are assessed separately. For instance “combination” encompasses means in which the first (or second or third) biomarker is assessed, wherein second (or first or third) biomarker may have been previously been assessed. The assessment of the three biomarkers may also be executed step-wise by the same or by different actors and by similar or by different techniques. For example, one actor may assess the first biomarker with one given technique, and a second actor may assess the second and/or third biomarker(s) by using the same or different technique(s). The assessment steps may be executed at the same time, or nearly the same time, or at distant times, in the same or in different sample(s) so long as it is possible to obtain a combined assessment on the subject. In a preferred embodiment, the three biomarkers are assessed simultaneously in the same tissue sample or in the same biopsy, or in the different biopsies within a relatively short period of time (i.e. less than 1 week).

C) Therapeutic Applications

Related aspects of the invention concern a method for preventing the progression of antibody mediated tissue injury in a subject with a sensitized allograft. In one embodiment the method comprises: measuring in the allograft endothelial cell expression of at least one biomarker (preferably a combination of two or three biomarkers) consisting of Fascin1, Vimentin and Hsp47, wherein the expression is indicative of an endothelial injury consecutive to binding and cellular consequences of donor specific antibodies (DSA) which have the property to target the endothelial cells and subsequently to engage a cellular response. Thus the expression of these endothelial biomarkers would help to identify the patients in whom reducing the level and the production of DSA is desirable or even mandatory in order to protect the allograft.

Reducing the level and the production of the DSA and/or protecting the allograft may be achieved using any suitable medical means known to those skilled in the art. In one embodiment, such reduction and protection comprise a therapeutic intervention with the subject such as administration of antithymomcy globulin (ATG), administration of monoclonal anti-CD20 antibodies (rituximab), administration of proteasome inhibitor (bortezomib), intravenous administration of immunoglobulins, plasmapheresis, administration of anti-05 antibodies (eculizumab) and splenectomy. Accordingly, the invention encompasses treatment methods wherein it becomes possible to block an allograft rejection or at least reduce aggravation of the rejection.

As used herein, “preventing” or “prevention” is intended to refer to at least the reduction of likelihood of the risk of (or susceptibility to) acquiring a disease or disorder (i.e. causing at least one of the clinical symptoms of the disease not to develop in a subject that may be exposed to or predisposed to the disease, but does not yet experience or display symptoms of the disease). Biological and physiological parameters for identifying such patients are provided herein and are also well-known by physicians and include presence in the plasma of patient's donor specific antibodies (DSA). More particularly, the methods of the invention may be useful in detecting the ABMR in an early phase (e.g. without evident clinical or morphological signs) and then preventing progression of tissue injury in antibody mediated rejection (ABMR) with a suitable intervention in an engrafted subject.

The terms “treatment” or “treating” of a subject includes the application or administration of a suitable compound, or composition of the invention as defined herein to a subject (or application or administration of a compound or composition of the invention to a cell or tissue from a subject) with the purpose of delaying, stabilizing, curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement, remission, slowing disease progression or severity, stabilization, diminishing of symptoms or making the injury, pathology or condition more tolerable to the subject, slowing in the rate of degeneration or decline, making the final point of degeneration less debilitating, or improving a subject's physical or mental well-being. In some embodiments, the term “treating” can include increasing a subject's life expectancy and/or delay before additional treatments are required.

### D) Kits

Given the correlation which exists between expression of the biomarkers Fascin1, Vimentin and Hsp47 and endothelial cell injury and/or activation, quantification of the expression of these three biomarkers may potentially detect any significant harm on the endothelium.

Accordingly, a related aspect of the invention relates to kits for diagnosing acute and/or chronic ABMR of an allograft, comprising a combination of antibodies specific for at least two biomarkers selected from Fascin1, Vimentin and Hsp47. In one embodiment, the kit comprises a combination of antibodies specific for each of Fascin1, Vimentin and Hsp47 (i.e. a combination of the different antibodies each specific for a given biomarker). According to the present invention, any kit comprising a combination of at least antibodies specific for Fascin1, Vimentin and Hsp47 is presumed to be a kit for use in diagnosing acute and/or chronic ABMR.

The kits of the invention may be particularly useful for applications in animals and humans according to the detection and/or diagnostic methods described hereinbefore. More particularly, the kits disclosed may be helpful for laboratory and diagnostic purposes in animals and humans: (i) for detecting endothelial cell injury and/or activation; (ii) for diagnosing acute and/or chronic antibody mediated rejection (ABMR) in an allograft; (iii) for identifying a subject showing endothelial injury related diseases (e.g. a vasculitis, a thrombotic microangiopathy, an antiphospholipid syndrome); and/or (iv) for preventing progression of antibody mediated tissue injury in a sensitized subject with an allograft or of other endothelial injury related diseases in a native and/or transplanted organ in mammalian subjects.

A kit of the invention may further comprise one or more of the following elements: paraffin slides containing the kidney tissue with the endothelial cell production of Fascin1, Vimentin and Hsp47 in peritubular capillaries to be used as positive controls; a buffer for target retrieval which may be used preceding the immunostaining; endogenous peroxidase blocking solution; incubation buffer(s), specific antibodies against fascin1, vimentin and hsp47; horseradish peroxidase (HRP) system for visualizing immunoreactions; a user manual, suitable components associated to detection of the biomarkers in plasma and/or urine, etc. Preferably the kit is optimized for immunohistochemistry and the kit further comprises components for immunohistochemistry visualization.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following example, which should not be construed as further or specifically limiting.

### EXAMPLES

The Examples set forth herein below provide exemplary methods and results showing detection of EndMT and diagnosis of ABMR in renal allografts. Also provided are exemplary methods for assessing expression of the biomarkers of the invention.

### Example 1

### Assessment of the Expression of Fascin1, Vimentin and Hsp47 in Human Renal Engrafted Patients

In the present example, 127 biopsies from 118 patients from a renal transplant center (Tenon hospital in Paris, France) were included in a study.

By immunohistochemistry, in the first setting of 53 biopsy, the expression of fascin1, vimentin and hsp47 in the endothelial cells of peri-tubular capillaries was studied and semi-quantified in 14 patients diagnosed clinically by physicians and treated as acute ABMR, plus 6 diagnosed with chronic ABMR. The expression was compared with that from control groups including 3 normal kidney samples obtained from the healthy part of kidneys removed because of a renal cancer, 9 allograft protocol biopsies showing a normal transplant, and 24 transplant biopsies with Cell Mediated Rejection (CMR).

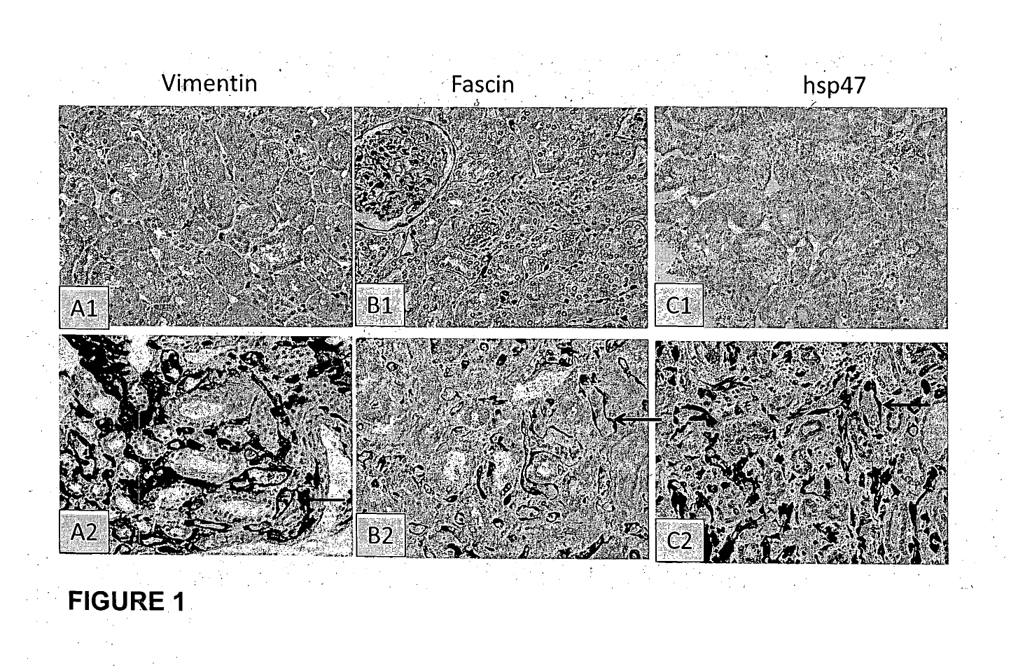

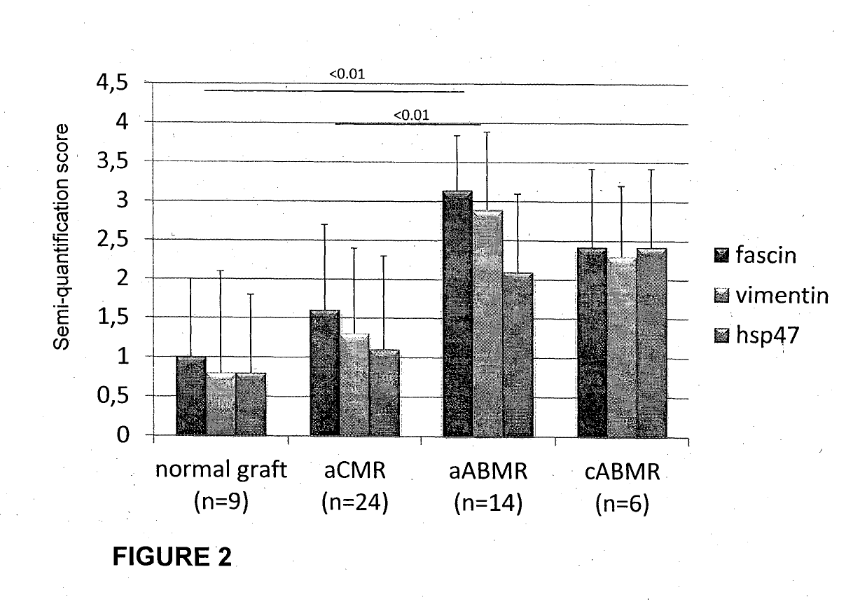

The results show an important and significant up-regulation of fascin1 and of de novo expression of vimentin and of hsp47 in the endothelial cells of pen-tubular capillaries in the patients with ABMR comparing to those in the control groups (FIG. 1 and FIG. 2). The level of expression of these EndMT markers was significantly correlated by a spearman's correlation test with the scores of micro vascular inflammation such as pen-tubular capillaritis (with rho=0.61, p<0.0001; rho=0.54, p<0.0001; rho=0.37, p=0.0083 for fascin1, vimentin and hsp47 respectively) and glomerulitis (with rho=0.5, p=0.0001; rtio=0.43, p=0.0012; rho=0.45, p=0.0009 for fascin1, vimentin and hsp47 respectively), Moreover the EndMT highest score was negatively correlated with the allograft function (with rho=−0.454, p=0.0006; rho=−0.43, p=0.0016; rho=−0.61, p<0.0001; rho=−0.634, p<0.0001; rho=−0.69, p<0.0001 for the estimated graft filtration rate (eGFR) at time of biopsy or one, two, three or four years post biopsy respectively) and positively with the abundance of proteinuria (with rho=0.5, p=0.0002; rho=0.31, p=0.0493; rho=0.6, p=0.0001; rho=0.341, p=0.06; rho=0.427, p=0.04 for the proteinuria at time of biopsy or one, two, three or four years post biopsy respectively).

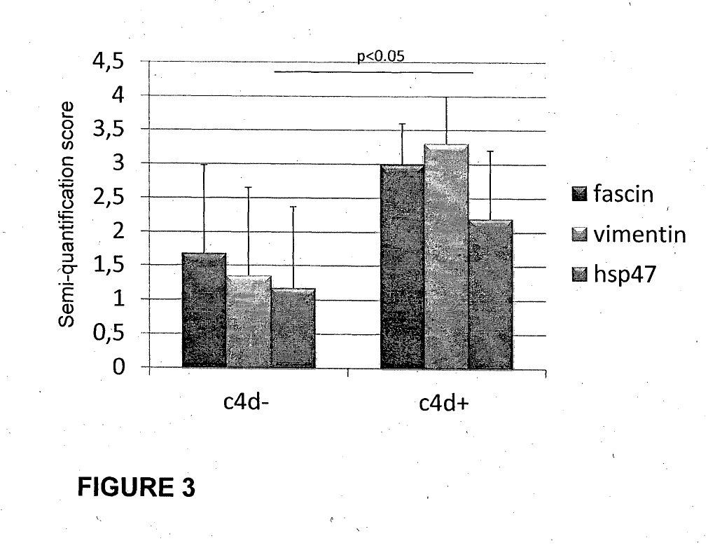

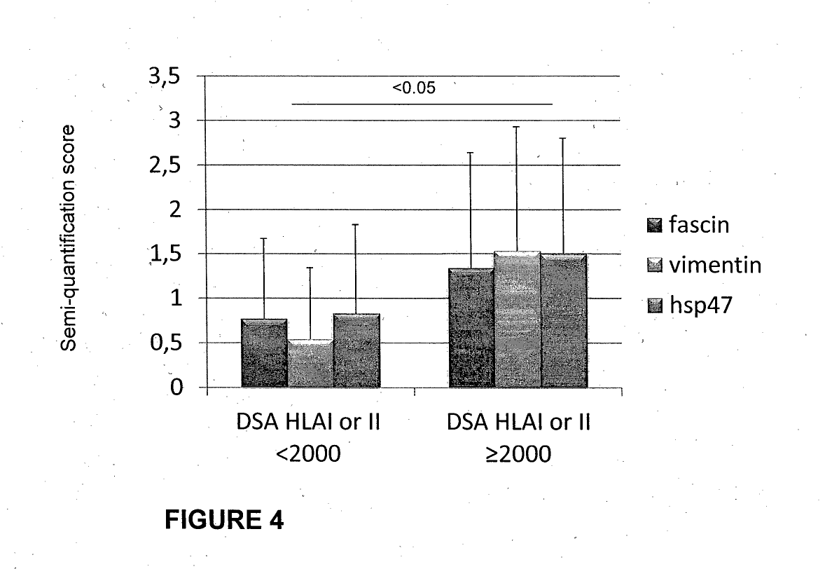

In addition, expression of these EndMT markers was significantly more intense in the allografts with C4d positive detected in pen-tubular capillaries than in that with C4d negative staining (m=3±0.6 vs 1.68±1.3, p=0.004; m=3.3±0.7 vs 1.35±1.3, p=0.0001; m=2.2±1 vs 1.17±1.2, p=0.026) (FIG. 3). Data obtained from a later experiment showed that the level of expression was also significantly higher in patients having anti-donor specific antibodies (DSA) in their plasma compared to those without (FIG. 4).

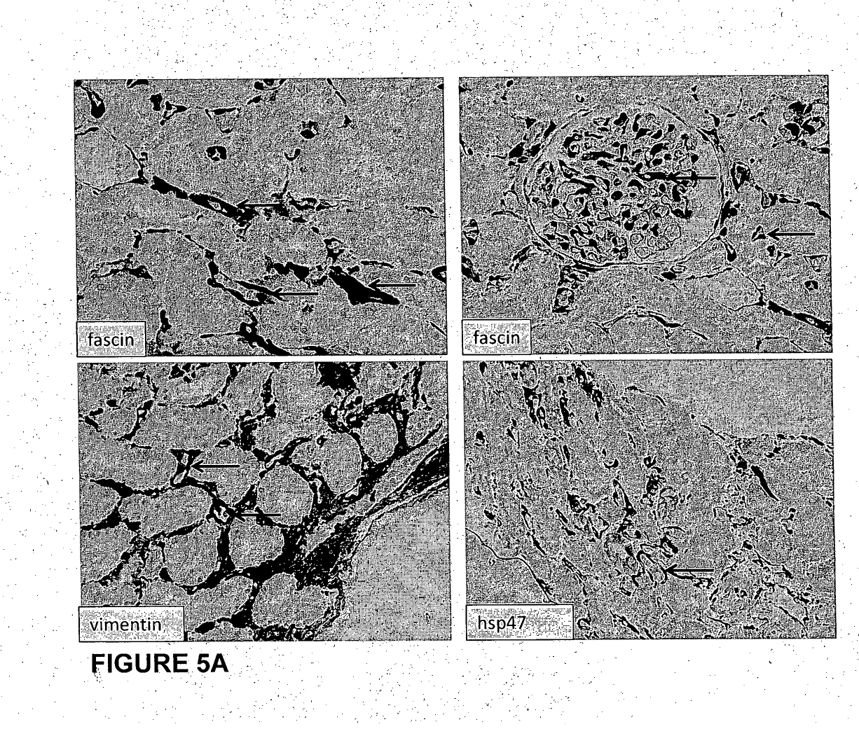

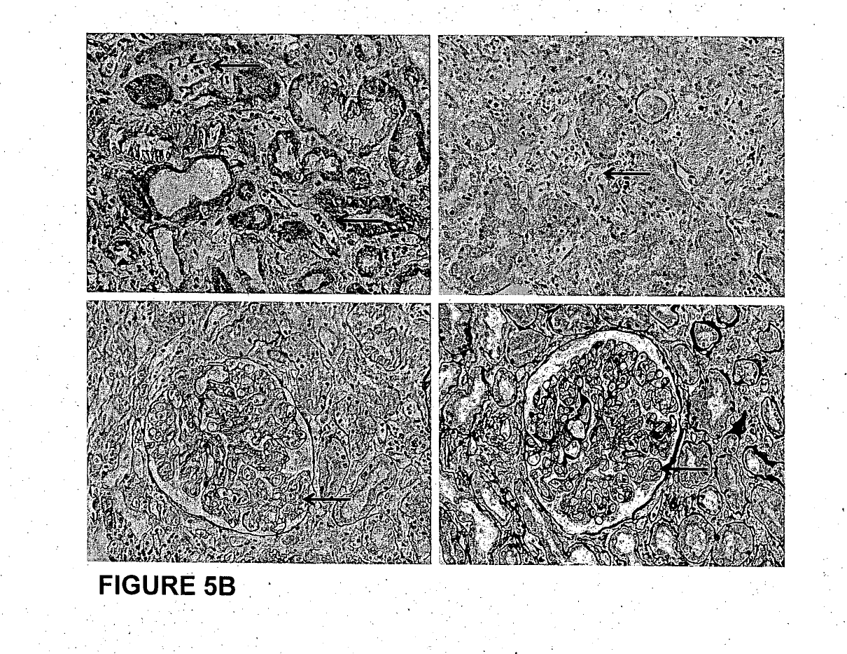

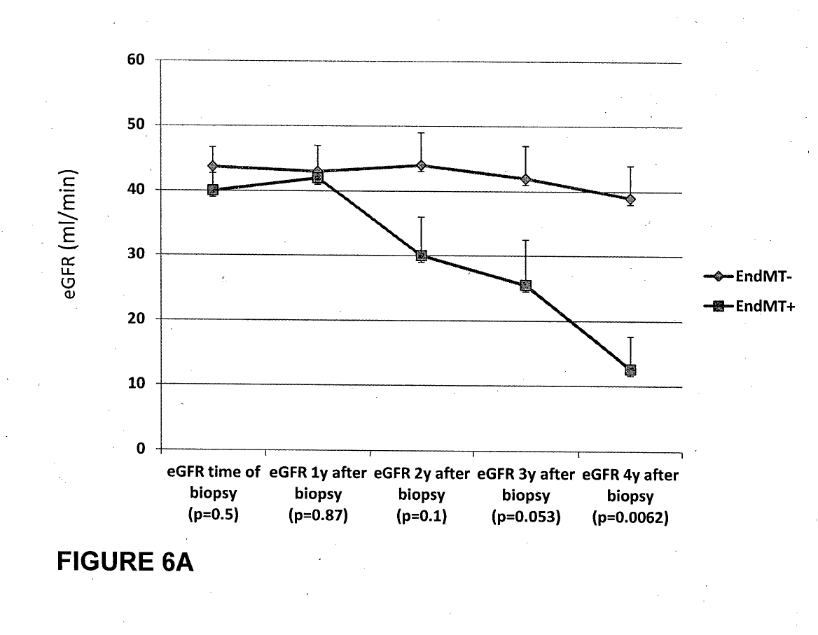

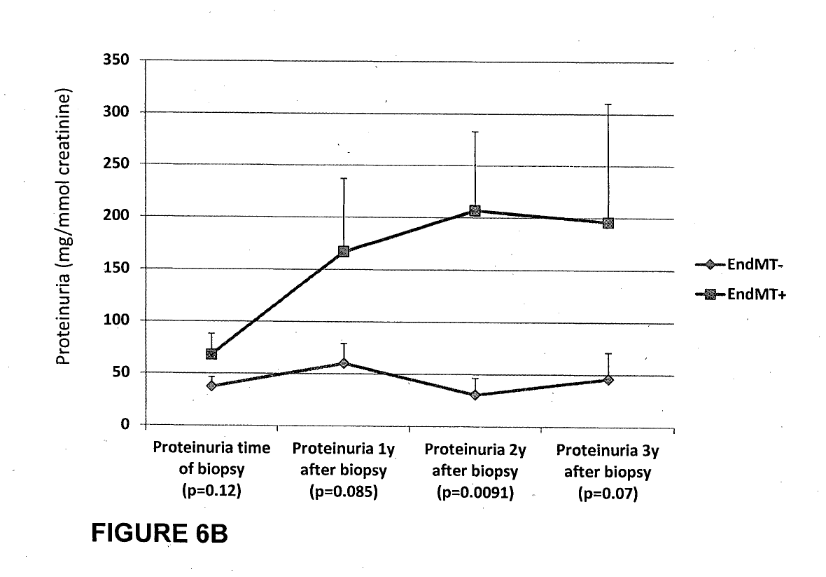

More interestingly, the EndMT markers were found to be more sensitive and more accurate compared to the current criteria for the diagnosis of ABMR. For example, the expression of EndMT markers was found in some patients who were not diagnosed as ABMR by the current Banff criteria at time of biopsy (11 patients diagnosed as CTMR and 2 considered as normal graft; FIG. 5A), suggesting unrecognized endothelial cell activation at the time of biopsy in these patients. Progressive loss of graft function and the occurrence of proteinuria, two important clinical events in patients with ABMR, were observed in these patients compared to those without EndMT marker expression who kept stable graft function up to 4 years post biopsy (FIG. 6A) although all patients (with or without EndMT expression) had an equal level of graft function at time of biopsy. Similar results were obtained for proteinuria (FIG. 6B). More convincingly, in the group of 13 patients expressing EndMT markers (FIG. 5A), six (6) patients had subsequent biopsy and ABMR was diagnosed in five (5) patients (FIG. 5B), whereas in the 20 patients without EndMT marker expression, eight (8) had subsequent biopsies and no patient was diagnosed as ABMR.

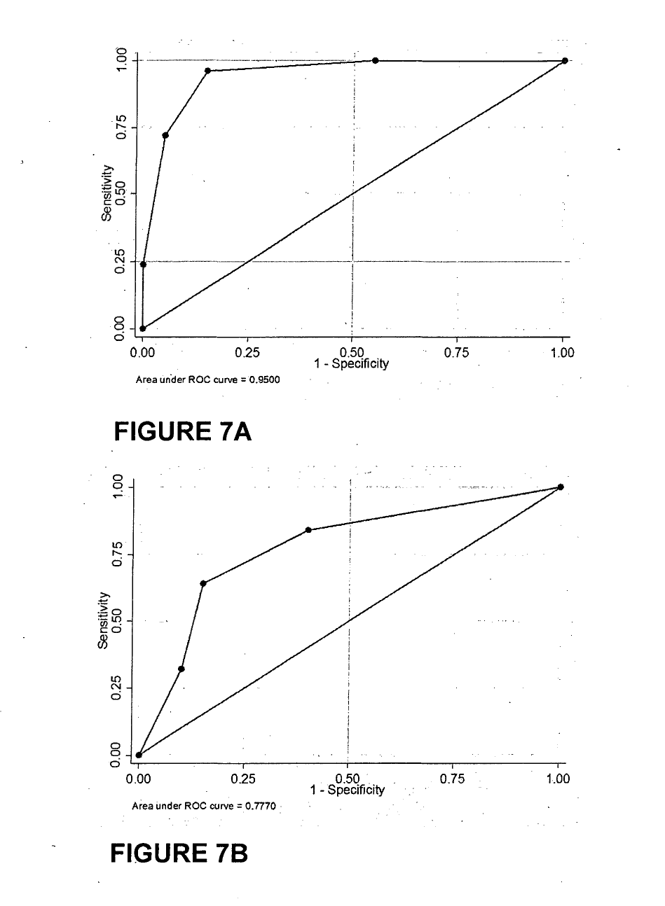

When we correct retrospectively the diagnosis of patients with the additional biopsies which demonstrated the ABMR lesions, ROC curve analysis showed a very good value of EndMT markers for ABMR diagnosis. The calculated area under the curve (AUC) was 0.95 [95% CI=0.89-1] (FIG. 7A). Using 2 as cutoff value which showed the best compromise of sensitivity (fraction of true positive results) and specificity (1—false positive results), ABMR was diagnosed with a sensitivity of 96% and a specificity of 85%; If we use 3 as cutoff value for EndMT score, ABMR will be diagnosed by EndMT markers with a sensitivity of 72% but a specificity of 95%. However, using ptc as the diagnostic tool, the AUC was only 0.78 [95% CI=0.64-0.915] (FIG. 7B) which gave a sensitivity of 84% and specificity of 60% when 1 was used as the cutoff value or a sensitivity of 64% and specificity of 85% if 2 was used as the cutoff value of ptc. The good diagnostic value of EndMT markers was also confirmed by a logistic regression model analysis with a LR chi2(4)=38.44, pseudo R2=0.72, p<0.0001, which revealed that EndMT marker expression was an independent risk factor associated with ABMR, with an odds ratio of 21.8 (95% CI=[1.97-241], p=0.012) after adjustment with ptc, HLA I antibodies and eGFR at time of biopsy. Moreover the likelihood-ratio test showed that the addition of EndMT markers into the logistic regression model including ptc, HLA I and eGFR at time of biopsy made the model fit significantly better for the diagnosis of ABMR, yielding a LR chi2 of 19.31 with p<0.0001. This test confirmed that EndMT was not only an independent but also necessary marker for improving ABMR diagnosis.

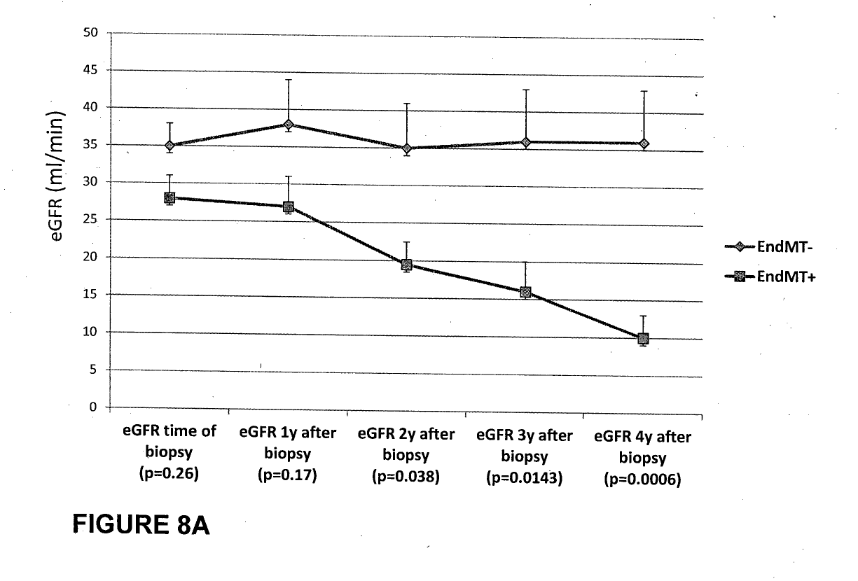

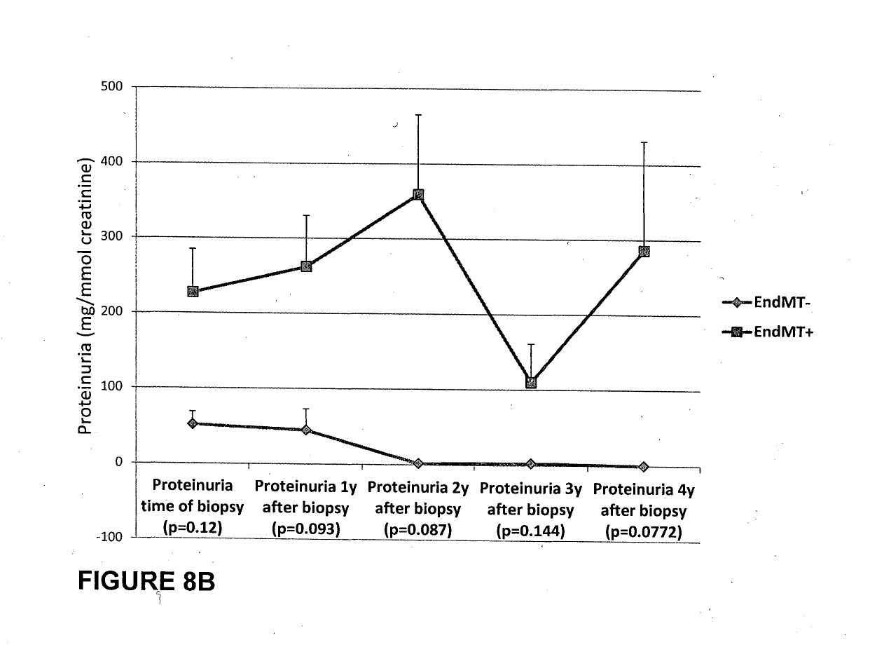

Considered currently as a more reliable diagnostic criterion of ABMR, peri-tubular capillaritis (ptc) has been shown to be closely associated with graft dysfunction and proteinuria in renal engrafted patients. However, in the patients with ptc, only those expressing EndMT markers at the time of biopsy deteriorated progressively their graft function, while for those who did not express EndMT markers, the graft function remained stable up to 4 years post transplantation (FIG. 8A). Similarly for proteinuria, in the patients with ptc, only those expressing EndMT markers had higher level of proteinuria up to 4 years post biopsy when compared with those without EndMT marker expression (FIG. 8B). The good predictive value of EndMT markers for long term graft function loss was confirmed by a logistic regression model analysis, which revealed that EndMT marker expression was an independent risk factor for a low eGFR prediction at 4 years post-biopsy, with an odds ratio of 12.63 (95% CI=1.086-146.81 p=0.043) after adjustment with ptc, C4d, CI and eGFR at time of biopsy. Moreover the likelihood-ratio test showed that the addition of EndMT markers into the logistic regression model including ptc, C4d and eGFR at time of biopsy made the model fit significantly better for predicting long term graft dysfunction, yielding a LR chi2 of 10.69 with p=0.0011. This test confirmed the value and the necessity of EndMT marker detection for graft surveillance. Comparing to C4d detection which was diffusely or focally positive only in 64% of ABMR, the markers of EndMT were positive in most of patients including those C4d-ABMR patients: when the score 2 was taken as cut off value, individually fascin1 was positive in 96%; vimentin was positive in 80% and hsp47 was positive in 68% of ABMR patients.

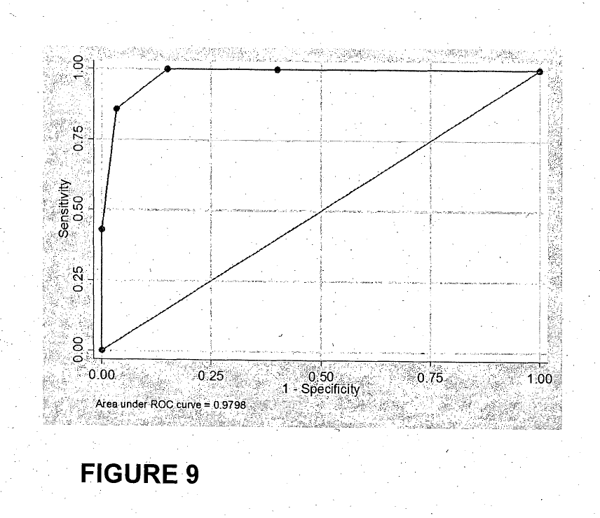

The diagnostic value of EndMT for ABMR was confirmed in a second independent setting of 74 renal transplant biopsies which showed a high level of EndMT marker expression in micro vascular endothelial cells in the patients with ABMR and the expression of these new markers was significantly associated with current ABMR diagnostic criteria such as microvascular inflammation, C4d peritubular capillary deposition and DSA. Again the graft dysfunction and proteinuria were associated with the EndMT marker expression. High sensitivity and specificity of EndMT markers for the diagnostic of ABMR was confirmed in this setting of biopsies by ROC curve analysis. The AUC was 0.98[95% CI=0.956-1] (FIG. 9) and ABMR was diagnosed by EndMT markers with a sensitivity of 100% and a specificity of 85% if 2 was used as the cutoff value of EndMT; or with a sensitivity of 85.7% and a specificity of 96.7% if 3 was used as the cutoff value.

Taken together, these results indicate EndMT detected by the endothelial expression of fascin1, vimentin and hsp47 is a direct proof for vascular endothelial cell activation which offers a more sensitive and more accurate marker for ABMR diagnosis. Therefore, combined assessment of fascin1, vimentin and hsp47 expression can be used as a new tool to diagnose ABMR in various types of allografts, particularly in renal allografts. Because the vascular endothelial cells are the major target of alloantibody and the mechanism of endothelial injury in allografts is indeed generic and not tissue-specific: in several non-kidney solid allografts such as the heart, lung, liver or the intestine, DSA will also lead to microvascular injury and cause the loss of allograft function.

These results also support the concept of therapeutic applications. For instance, expression of fascin1, vimentin and hsp47 can serve as an indicator of an endothelial injury consecutive to binding of donor specific antibodies (DSA) which will engage a cellular response by the endothelium, and whenever necessary, the therapeutic process can be introduced in an early stage such as to reduce the level of alloantibodies, to stop the production of DSA and to protect the allograft against the antibody mediated tissue injury.

### Example 2

### Diagnosis of Antibody Mediated Rejection (ABMR) in Engrafted Human Patients

In clinic, antibody mediated rejection (ABMR) is disclosed by the biopsy of patients mostly for cause i.e. because of the deterioration of their graft function or of proteinuria, however, few cases can be found by surveillance biopsies i.e. without alteration of graft function. In such biopsies, when micro-circulation inflammation such as glomerulitis or capillaritis is observed along with deposition of C4d in the peritubular capillaries in the presence of donor specific antibodies (DSA) to HLA antigen in recipient's plasma, the diagnosis of antibody mediated rejection is established. However, in the practice, we often meet some atypical cases, who do not fulfill these 3 criteria for antibody mediated rejection (ABMR) as defined by the international Banff group,

For example, a kidney recipient, engrafted three months earlier and known to express donor specific anti HLA antibodies, undergoes a surveillance biopsy (no allograft dysfunction) that discloses some degree of capillaritis, yet the C4d staining is negative, leaving the clinicians with a doubtful diagnosis of antibody mediated rejection.

In this case, the invention would offer a direct proof of endothelial (major target of alloantibodies) injury and/or activation thus supporting the diagnosis of DSA-mediated allograft injury and hence antibody-mediated rejection, would the EndMT markers stain positive.

Another example: EndMT markers will stain positive in patients with DSA yet no notable or low grade capillaritis but without capillary dilation, as early biomarkers of antibody-mediated rejection. This would further increase the interest in these markers. This is the case referred in FIG. 5, and need to be confirmed in a population of DSA positive patients with no or low grade capillaritis without capillary dilation, but with positive EndMT markers, to determine whether they were diagnosed with antibody-mediated rejection at later time points.

### Example 3

### Non-Invasive Detection

It is conceivable according to the present invention to use non-invasive methods for the detection of EndMT markers, including but not limited to detection in the blood and/or urine of an engrafted recipient.

For instance, the upregulated expression of fascin1, vimentin and/or hsp47 by vascular endothelial cells during ABMR could be further detected in the plasma and/or in the urine using suitable detection methods of proteins such as Enzyme-linked immunosorbent assay (ELISA) or immunoblottings.

Upregulated expression of any of the three biomarkers could also be detected by RT-PCR, flow cytometric analysis or the like in detached vascular endothelia cells or in the microparticules shed from the broken endothelial cells that may be present the blood of an engrafted recipient or of patients with microvascular endothelial activation related diseases.

Because of their non-invasive nature such methods could be repeated frequently for allowing a continuous monitoring of the subject's condition and continuous risk assessment of graft rejection. The biomarker(s) expression obtained by using such non-invasive methods may subsequently be validated with a biopsy, it may guide the subsequent biopsy (e.g. selection of the tissue) and it may also improve further graft surveillance.

### Example 4

### Prevention of Progression of Antibody Mediated Graft Injury in Engrafted Human Patients

The present invention will help to identify the early ABMR patients (e.g. a patient according to Example 2 who is DSA positive, C4d negative, with minor or no inflammation in microcirculation such as glomerulitis or capillaritis but EndMT positive) and then introduce the treatment for blocking allograft rejection or at least reduce aggravation of the rejection. The patient is treated using one or more protocol(s) aiming at the eradication of DSA, aiming at the reduction in the synthesis of DSA and/or aiming at protecting the allograft against complement triggered allograft injury. The treatment protocol(s) comprise plasmapheresis, administration of IgIV, administration of anti-CD20 or antithymocyte antibodies, administration of complement activation inhibitor (eculizumab), administration of compounds for inactivating the complement (e.g. anti-05 antibodies) and/or administration of proteasome inhibitors (e.g. bortezomib). For additional comfort, the combined EndMT markers are monitored during the course of the treatment and they are used as efficacy indicators of the treatment.

### REFERENCES

1. Gaston R S, Cecka J M, Kasiske B L, Fieberg A M, Leduc R, Cosio F C, Gourishankar S, Grande J, Halloran P, Hunsicker L, Mannon R, Rush D, Matas A J: Evidence for antibody-mediated injury as a major determinant of late kidney allograft failure. Transplantation 2010; 90:68-74.
2. Einecke G, Sis B, Reeve J, Mengel M, Campbell P M, Hidalgo L G, Kaplan B, Halloran P F: Antibody-mediated microcirculation injury is the major cause of late kidney transplant failure. Am J Transplant 2009; 9:2520-2531.
3. EI-Zoghby Z M, Stegall M D, Lager D J, Kremers W K, Amer H, Gloor J M, Cosio F G: Identifying specific causes of kidney allograft loss. Am J Transplant 2009; 9:527-535.
4. Jin Y P, Singh R P, Du Z Y, Rajasekaran A K, Rozengurt E, Reed E F: Ligation of HLA class I molecules on endothelial cells induces phosphorylation of Src, paxillin, and focal adhesion kinase in an actin-dependent manner. J Immunol 2002; 168:5415-5423.
5. Lepin E J, Zhang Q, Zhang X, Jindra P T, Hong L S, Ayele P, Peralta M V, Gjertson D W, Kobashigawa J A, Wallace W D, Fishbein M C, Reed E F: Phosphorylated S6 ribosomal protein: a novel biomarker of antibody-mediated rejection in heart allografts. Am J Transplant 2006; 6:1560-1571.
6. Jindra P T, Hsueh A, Hong L, Gjertson D, Shen X D, Gao F, Dang J, Mischel P S, Baldwin W M, 3rd, Fishbein M C, Kupiec-Weglinski J W, Reed E F: Anti-MHC class I antibody activation of proliferation and survival signaling in murine cardiac allografts. J Immunol 2008; 180:2214-2224.
7. Ziegler M E, Souda P, Jin Y P, Whitelegge J P, Reed E F: Characterization of the endothelial cell cytoskeleton following HLA class I ligation. PLoS One 2012; 7:e29472.
8. Sis B, Jhangri G S, Bunnag S, Allanach K, Kaplan B, Halloran P F: Endothelial gene expression in kidney transplants with alloantibody indicates antibody-mediated damage despite lack of C4d staining. Am J Transplant 2009; 9:2312-2323.
9. Hertig A, Verine J, Mougenot B, Jouanneau C, Ouali N, Sebe P, Glotz D, Ancel P Y, Rondeau E, Xu-Dubois Y C: Risk factors for early epithelial to mesenchymal transition in renal grafts. Am J Transplant 2006; 6:2937-2946.
10. Hertig A, Anglicheau D, Verine J, Pallet N, Touzot M, Ancel P Y, Mesnard L, Brousse N, Baugey E, Glotz D, Legendre C, Rondeau E, Xu-Dubois Y C: Early epithelial phenotypic changes predict graft fibrosis. J Am Soc Nephrol 2008; 19:1584-1591.
11. Xu-Dubois Y C, Baugey E, Peltier J, Colombat M, Ouali N, Jouanneau C, Rondeau E, Hertig A: Epithelial phenotypic changes are associated with a tubular active fibrogenic process in human renal grafts. Hum Pathol 2013.
12. Zeisberg E M, Tarnayski O, Zeisberg M, Dorfman A L, McMullen J R, Gustafsson E, Chandraker A, Yuan X, Pu W T, Roberts A B, Neilson E G, Sayegh M H, Izumo S, Kalluri R: Endothelial-to-mesenchymal transition contributes to cardiac fibrosis. Nat Med 2007; 13:952-961.
13. Zeisberg E M, Potenta S E, Sugimoto H, Zeisberg M, Kalluri R: Fibroblasts in kidney fibrosis emerge via endothelial-to-mesenchymal transition. J Am Soc Nephrol 2008; 19:2282-2287.
14. Zeisberg E M, Kalluri R: Origins of cardiac fibroblasts. Circ Res 2010; 107:1304-1312.
15. Hashimoto Y, Kim D J, Adams J C: The roles of fascins in health and disease. J Pathol 2011; 224:289-300.
16. Vuoriluoto K, Haugen H, Kiviluoto S, Mpindi J P, Nevo J, Gjerdrum C, Tiron C, Lorens J B, Ivaska J: Vimentin regulates EMT induction by Slug and oncogenic H-Ras and migration by governing Axl expression in breast cancer. Oncogene 2011; 30:1436-1448.
17. Taguchi T, Razzaque M S: The collagen-specific molecular chaperone HSP47: is there a role in fibrosis? Trends Mol Med 2007; 13:45-53.
18. Razzaque M S, Taguchi T: Collagen-binding heat shock protein (HSP) 47 expression in anti-thymocyte serum (ATS)-induced glomerulonephritis. J Pathol 1997; 183:24-29.
19. Rastaldi M P, Ferrario F, Giardino L, Dell'Antonio G, Grillo C, Grillo P, Strutz F, Muller G A, Colasanti G, D'Amico G: Epithelial-mesenchymal transition of tubular epithelial cells in human renal biopsies. Kidney Int 2002; 62:137-146.
20. Mahesh B, Leong H S, McCormack A, Sarathchandra P, Holder A, Rose M L: Autoantibodies to vimentin cause accelerated rejection of cardiac allografts. Am J Pathol 2007; 170:1415-1427.
21. Ohba K, Miyata Y, Koga S, Nishikido M, Kanetake H, Nazneen A, Razzaque M S, Taguchi T: Interstitial expression of heat-shock protein 47 correlates with capillary deposition of complement split product C4d in chronic allograft nephropathy. Clin Transplant 2005; 19:810-816.
22. Mengel M, Sis B, Haas M, Colvin R B, Halloran P F, Racusen L C, Solez K, Cendales L, Demetris A J, Drachenberg C B, Farver C F, Rodriguez E R, Wallace W D, Glotz D: Banff 2011 Meeting Report New Concepts in Antibody-Mediated Rejection. Am J Transplant 2012; 12:563-570.
23. Zou Y, Stastny P, Susal C, Dohler B, Opelz G: Antibodies against MICA antigens and kidney-transplant rejection. N Engl J Med 2007; 357:1293-1300.
24. Jordan S C, Yap H K, Sakai R S, Alfonso P, Fitchman M: Hyperacute allograft rejection mediated by anti-vascular endothelial cell antibodies with a negative monocyte crossmatch. Transplantation 1988; 46:585-587.
25. Dragun D, Muller D N, Brasen J H, Fritsche L, Nieminen-Kelha M, Dechend R, Kintscher U, Rudolph B, Hoebeke J, Eckert D, Mazak I, Plehm R, Schonemann C, Unger T, Budde K, Neumayer H H, Luft F C, Wallukat G: Angiotensin II type 1-receptor activating antibodies in renal-allograft rejection. N Engl J Med 2005; 352:558-569.
26. Loupy A, Hill G S, Suberbielle C, Charron D, Anglicheau D, Zuber J, Timsit M O, Duong J P, Bruneval P, Vernerey D, Empana J P, Jouven X, Nochy D, Legendre C H: Significance of C4d Banff scores in early protocol biopsies of kidney transplant recipients with preformed donor-specific antibodies (DSA). Am J Transplant 2011; 11:56-65.
27. Sis B, Jhangri G S, Riopel J, Chang J, de Freitas D G, Hidalgo L, Mengel M, Matas A, Halloran P F: A New Diagnostic Algorithm for Antibody-Mediated Microcirculation Inflammation in Kidney Transplants. Am J Transplant 2012.

Headings are included herein for reference and to aid in locating certain sections. These headings are not intended to limit the scope of the concepts described therein under, and these concepts may be applicable in other sections throughout the entire specification. Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a biomarker” includes one or more of such biomarkers, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and so forth.

It is understood that the examples and embodiments described herein are for illustrative purposes only and that various modifications or changes in light thereof will be suggested to persons skilled in the art and are to be included within the present invention and scope of the appended claims.

1. A method for detecting endothelial cell injury and/or activation in a mammalian subject, comprising detecting endothelial to mesenchymal transition (EndMT), wherein presence of EndMT is indicative of cell injury and/or activation.

2. The method of claim 1, wherein detecting EndMT comprises assessing expression of at least one biomarker selected from the group consisting of Fascin1, Vimentin and Hsp47.

3. The method of claim 1, wherein detecting EndMT comprises assessing expression of at least two biomarkers selected from the group consisting of Fascin1, Vimentin and Hsp47.

4. The method of claim 1, wherein detecting EndMT comprises assessing expression of a combination of three biomarkers consisting of Fascin1, Vimentin and Hsp47.

5. The method of any one of claims 1 to 4, wherein said endothelial cell injury and/or activation is detected in an allograft in a sensitized subject and is indicative of an antibody mediated rejection (ABMR) of said allograft after excluding other endothelial activation related diseases such as recurrent thrombotic microangiopathy.

6. The method of claim 5, wherein said ABMR is detected in a renal allograft, a heart allograft, a lung allograft, a liver allograft, a pancreas allograft and/or an intestine allograft.

7. The method of claim 5, wherein said allograft is a solid organ and wherein said endothelial cell injury and/or activation is indicative of a vasculitis, of a thrombotic microangiopathy and/or of an anti-phospholipid syndrome.

8. The method of any one of claims 1 to 4, wherein said mammalian subject has received an allograft, and wherein detection of endothelial cell injury and/or activation is carried out to assess a potential acute and/or chronic humoral rejection of the allograft.

9. The method of claim 8, wherein said allograft is selected from the group consisting of a renal allograft, a heart allograft, a lung allograft, a liver allograft, a pancreas allograft, an intestine allograft, a body member allograft, a muscle allograft, and a face engraft.

10. The method of any one of claims 1 to 4, wherein said mammalian subject has received a renal allograft and wherein expression of said biomarker(s) is (are) assessed in peritubular capillaries and/or in glomerulus of the renal allograft.

11. The method of any one of claims 1 to 10, wherein the mammalian subject is a human patient.

12. The method of any one of claims 1 to 11, wherein the mammalian subject is an engrafted human patient.

13. The method of any one of claims 1 to 12, comprising assessing expression of at least one biomarker selected from the group consisting of Fascin1, Vimentin and Hsp47 in urine and/or plasma of a human patient.

14. A method for diagnosing antibody mediated rejection (ABMR) in an allograft, comprising assessing expression level of at least one biomarker selected from the group consisting of Fascin1, Vimentin and Hsp47, wherein said expression level is indicative of the presence or absence of ABMR in said allograft.

15. The method of claim 14, wherein detecting EndMT comprises assessing expression of at least two of said biomarkers.

16. The method of claim 14, wherein detecting EndMT comprises assessing expression of a combination of three biomarkers consisting of Fascin1, Vimentin and Hsp47.

17. The method of any one of claims 14 to 16, wherein assessing expression level of said biomarker(s) comprises obtaining a biopsy from said allograft.

18. The method of any one of claims 14 to 17, wherein increased expression level for each of Fascin1, Vimentin and Hsp47 when compared to a control value is indicative of the presence of ABMR lesions in said allograft.

19. The method of claim 18, wherein said control value comprises expression levels in subjects without endothelial cells injury-related disease.

20. The method of any one of claims 14 to 19, wherein presence of ABMR lesions in said allograft is indicative of presence of an acute and/or chronic ABMR of the allograft.

21. The method of any one of claims 14 to 20, wherein said allograft is selected from the group consisting of a renal allograft, a heart allograft, a lung allograft, a liver allograft, a pancreas allograft, an intestine allograft, a body member allograft, a muscle allograft, and a face engraft.

22. The method of claim 21, wherein said allograft is a renal allograft and wherein said expression level of said biomarker(s) is(are) assessed in peritubular capillaries and/or in glomerulus of the renal allograft.

23. The method of any one of claims 14 to 22, further comprising assessing at least one of peri-tubular capillaritis (ptc), deposition of C4d, estimated graft filtration rate (eGFR) and proteinuria.

24. The method of any one of claims 14 to 23, comprising assessing expression of at least one biomarker selected from the group consisting of Fascin1, Vimentin and Hsp47 in urine and/or plasma of a human patient.

25. A method for identifying a mammalian subject showing endothelial injury comprising assessing in endothelial cells expression level of at least one biomarker selected from the group consisting of Fascin1, Vimentin and Hsp47, wherein said expression level is indicative of the presence or absence of endothelial injury.

26. The method of claim 25, wherein detecting EndMT comprises assessing expression of at least two biomarkers.

27. The method of claim 25, wherein detecting EndMT comprises assessing expression of a combination of three biomarkers consisting of Fascin1, Vimentin and Hsp47.

28. The method of any one of claims 25 to 27, wherein said endothelial injury is consecutive to an event selected from the group consisting of: production of donor specific antibodies (DSA), a virus infection, a toxin aggression, coagulation problem, autoimmune disorders.

29. The method any one of claims 25 to 28, wherein the mammalian subject is an engrafted human patient in whom production of donor specific antibodies (DSA) has been detected.

30. The method of claim 29, wherein said engrafted human patient has received an allograft selected from the group consisting of: a renal allograft, a heart allograft, a lung allograft, a liver allograft, a pancreas allograft, and/or an intestine allograft.

31. The method of claim 28, wherein the mammalian subject is a non-grafted human patient having a virus infection, a toxin aggression, a coagulation problem or an autoimmune disorder.

32. A method for preventing progression of antibody mediated tissue injury in a patient with an allograft, comprising:
measuring in the allograft of the patient expression of at least one biomarker selected from the group consisting of Fascin1, Vimentin and Hsp47, wherein said expression is indicative of an endothelial injury consecutive to binding of donor specific antibodies (DSA) to endothelial cells; and
reducing the level and/or the production of said DSA.

33. The method of claim 32, wherein detecting EndMT comprises assessing expression of at least two of said biomarkers.

34. The method of claim 32, wherein detecting EndMT comprises assessing expression of a combination of three biomarkers consisting of Fascin1, Vimentin and Hsp47.

35. The method of any one of claims 32 to 34, further comprising protecting the allograft against antibody mediated tissue injury.

36. The method of any one of claims 32 to 35, wherein reducing the level and/or production of said DSA comprises a therapeutic intervention selected from the group consisting of: administration of monoclonal anti-CD20, proteasome inhibitor (bortezomib), administration of polyclonal antithymocyte antibodies, intravenous administration of immunoglobulins, plasmapheresis.

37. The method of any one of claims 32 to 36, further comprising administering anti-05 antibodies.

38. The method of any one of claims 32 to 37, wherein measuring expression of said biomarker(s) comprises obtaining a biopsy from said allograft.

39. A diagnostic kit, comprising a combination of antibodies specific for at least two biomarkers selected from the group consisting of Fascin1, Vimentin and Hsp47.

40. The kit of claim 39, wherein said kit comprises a combination of antibodies specific for each of Fascin1, Vimentin and Hsp47.

41. The kit of claim 39 or 40, wherein said kit is optimized for immunohistochemistry and wherein the kit further comprises components for immunohistochemistry visualization.

42. The kit of any one of claims 39 to 41, wherein said kit further comprises instructions for diagnosing in a human patient endothelial to mesenchymal transition (EndMT) during acute and/or chronic antibody mediated rejection (ABMR) of an allograft.

43. Use of at least one biomarker selected from the group consisting of Fascin1, Vimentin and Hsp47: (i) for detecting endothelial cell injury and/or activation in a mammalian subject; (ii) for diagnosing acute and/or chronic ABMR of an allograft in a mammalian subject; (iii) for identifying a mammalian subject showing endothelial injury related diseases; and/or (iv) for preventing the progression of antibody mediated rejection (ABMR) in a sensitized subject with an allograft.
